(19) United States
(12) Patent Application Publication
(10) Pub. No.: US 2009/0187158 A1
(43) Pub. Date: Jul. 23, 2009

(54) CONVERSION DEVICE

(76) Inventor: Frank M. Richmond, Harvard, IL (US)

Correspondence Address: MCDERMOTT, WILL & EMERY, 11682 EL CAMINO REAL, SUITE 400, SAN DIEGO, CA, 92130-2047, US

(21) Appl. No.: 12/090,834

(22) PCT Filed: Oct. 19, 2006
(86) PCT No.: PCT/US06/40896

§ 371 Date: Mar. 9, 2009

Related U.S. Application Data
(60) Provisional application No. 60/728,480, filed on Oct. 20, 2005.
(60) Provisional application No. 60/742,314, filed on Dec. 5, 2005.

(51) Int. Cl. A61J 1/14

(52) U.S. Cl. 604/414; 604/415

(57) ABSTRACT

A conversion device is provided that is effective for converting/adapting a spiked medical device/disposable to a valve/port on a container, such as for example a reflux-valve port on a container. The conversion device includes an attachment feature, at least one membrane port usually on an end opposite the attachment feature, and an intermediate structure disposed between the attachment feature and membrane port.

The present invention relates to a conversion device. More particularly, a conversion device is provided that is effective for converting or adapting a spiked medical device/disposable to a valve on a container.

### BACKGROUND

Fluid contained in intravenous fluid bags or containers used in the medical field have traditionally been administered using a spike. A user inserts the spike through a membrane port on the bag or container which allows fluids to flow out of the bag. In a significant number of uses, the spike is accidentally put through the bag. This accidental spiking of the bag renders the contents of the bag useless, may result in contamination of the environment from escape of fluids from the bag, and may result in contamination of the health care worker.

In an effort to address problems associated with the use of a spike, the trend in the medical disposable field is for containers to have either male or female reflux valves added as a means of access to the containers. Some examples of these types of containers and valves are described in U.S. Pat. Nos. 5,391,150, 5,405,333, 5,645,538, 6,068,617, 6,485,472, CA 2,171,857 and EP0719158.

While the trend in the medical disposable field is toward the use of containers with valves, container which can only be accessed with spikes will still be in use. On occasion, a user may want to attach a medical device/disposable with a spike to the container that includes male or female reflux valves. In that situation a user will need a conversion device that allows for a connection.

### SUMMARY

A conversion device is provided that is effective for converting/adapting a spiked medical device/disposable to a valve/port on a container, such as for example a reflux-valve port on a container. The conversion device includes an attachment feature, at least one membrane port usually on an end opposite the attachment feature, and an intermediate structure disposed between the attachment feature and membrane port. The attachment feature may be at least one of a male connector, female connector, cannula, male luer, male luer and collar, split skirt, female luer, bayonet connector, threads, partial threads, tabs, ears, male reflux valve, female reflux valve, goal post style connectors, flow restriction valve, or one-way valve, or valves of any design. The intermediate structure may include containers, fluid lines, tubing, connectors, sets, kits, drip chambers, filters, burette chambers, stopcocks, multiport valves, vials, flow restriction valve, ‘Y’-sites and ‘T’-sites, sets (for example gravity sets, pump sets, specialty sets and the like), and ambulatory pumps, or valves of any design. The conversion device may further include an injection site.

In another aspect, a method is provided for connecting a spike to a valve/port or reflux valve container. The method includes connecting a valve to an attachment feature on a conversion device and spiking a membrane port on the conversion device with a spike of the spiked medical system. The spiked medical system may include, for example, a set, pump set, gravity set, and ambulatory pump. The attachment feature may be at least one of male connector, female connector, cannula, male luer, male luer and collar, split skirt, female luer, bayonet connector, threads, partial threads, tabs, ears, male reflux valve, female reflux valve, goal post style connectors, flow restriction valve, or one-way valve, or valve of any design.

### BRIEF DESCRIPTION OF THE DRAWINGS

FIG. 1 generally describes attachment features in combination with an intermediate structure and a membrane port on another port.

FIG. 2 shows an adaptor with a female luer distal end and a membrane port proximal end.

FIG. 3 shows an adaptor with a male luer distal end and a membrane port proximal end.

FIG. 4 illustrates an adaptor with a male distal end with a bayonet attachment feature and a membrane port proximal end.

FIG. 5 shows a female-reflux valve (FRV) distal end and a membrane port proximal end.

FIG. 6 illustrates a male-reflux valve (MRV) distal end and a membrane port proximal end.

FIG. 7 shows a container with two ports, one with a FRV and the other with a membrane port plus an connector/adaptor with a male luer adaptor with a membrane port proximal end.

FIG. 8 shows a connector/adaptor with a female leur distal end and membrane port proximal end and a container with two ports. One port is a FRV and the other a MRV.

FIG. 9 illustrates a multi-layered container with at least one compartment and at least one port.

FIG. 10 shows a container with two ports. One showing a bayonet connector and an adaptor with a bayonet connector on the distal end and a membrane port on the proximal end.

FIG. 11 shows a container with a port, an adaptor with MRV on the distal end and membrane on the proximal end along with a spiked drip-chamber and set.

FIG. 12 illustrates a container with a MRV and a adaptor with a FRV distal end and a membrane port proximal end.

FIG. 13 shows an adaptor with a male-luer distal end and at least two membranes proximal ends along with a set.

FIG. 14 illustrates a connector/adaptor with a male connector on its distal end with at least one membrane port proximal and at least one other port that includes a reflux valve.

FIG. 15 shows a container with a MRV and an adaptor with two membrane ports at proximal ends along with an injection port.

FIG. 16 shows a container with a split septum port and an adaptor with goal-post attachment on the distal end and membrane port proximal end.

FIG. 17 shows a dual port container with a split septum port and an adaptor with goal-post attachment on the distal end and membrane port proximal end.

FIG. 18 shows a drip chamber with a drip chamber cap with a threaded means of attachment and cannula with an adaptor with a membrane port.

FIG. 19 shows an adaptor with a male leur distal end, a membrane port proximal end, and a clamp.

FIG. 20 illustrates a conversion device with a flexible skirt connector and cannula.

### DETAILED DESCRIPTION

The connectors illustrated in the drawings show various designs. It is understood that any person skilled in the art can vary the attachment feature and intermediate structure in connection with at least one membrane port in a number of different ways. Ports, connectors and various intermediate structures may be attached to each other using methods known in the art such as for example, solvent bonding, rf welding, ultrasonic welding, spin welding, insertion molded, and other means known in the art. The conversion device may be completely or in part be made out of materials known in the art, such as for example, plastics, metal, glass and any other known materials.

The attachment feature and intermediate structure may also include flow restriction valves. Examples of flow restriction valves include duck bills, umbrella, spring loaded balls, spring loaded plugs and any other flow restriction valves known in the art.

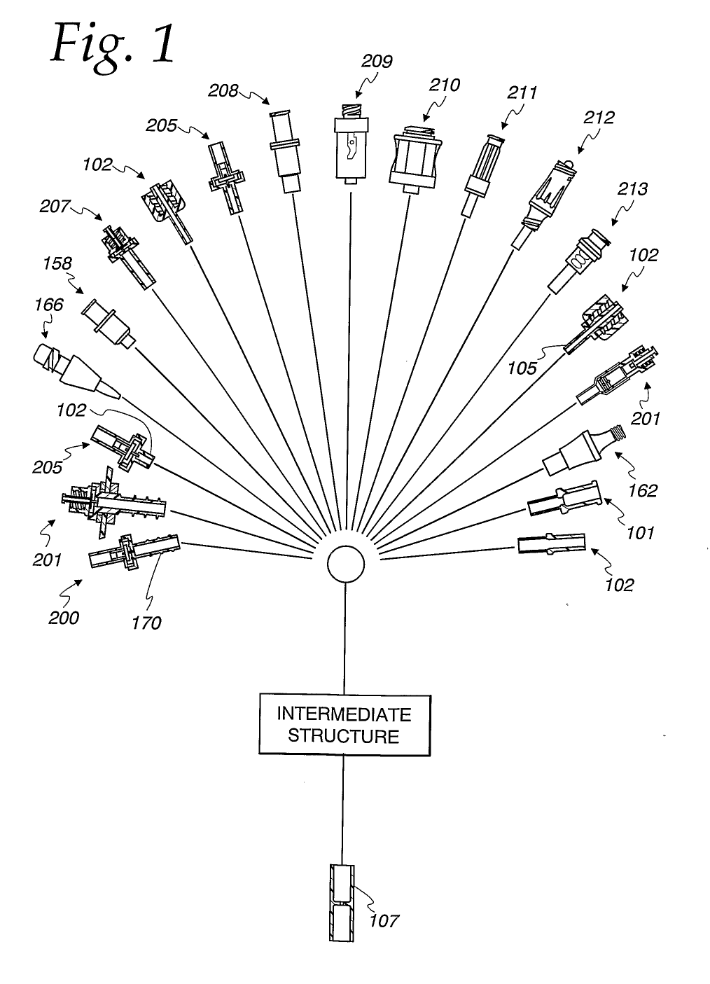

As generally shown in FIG. 1, the conversion device may include any number of attachment features with an intermediate structure and a membrane port 107. As used herein, “attachment feature” may include for example a female connector 200 with barbed shaft 170, a male connector 201, female reflux valve 205 that includes a male luer 102, split skirt 610, an interlink style connector 166 (Baxter), Halkey Roberts female valve 158, male reflux valve 207, male luer 102, female refulux valve 205, Filtertech swabable female reflux valve 208, Porex swabable female reflux valve 209, Halkey Roberts swabable female reflux valve 210, Braun swabable female reflux valve 211, Alaris male reflux valve 212, Alaris swabable female reflux valve 213, male luer 102 with rotatable collar 105, Clave reflux valve 162, female luer 101, a male luer 102, one-way valve (not shown), cannula (not shown), goal post connectors (not shown) and international equivalents of the attachment features.

One example of valves that may be utilized includes valves that include a resilient valve member. In this aspect, the resilient valve member defines an outer periphery that is uninterrupted within the periphery. The valve member is deformable to a configuration wherein fluid communication is permitted. In another related aspect, the valve may include a valve member defining an outer periphery that is interrupted at least once within the periphery. The interruption within the periphery allows the fluid to pass directly through the member.

The “intermediate structure” referred to in FIG. 1 may include, for example, containers, fluid lines, tubing, connectors, sets, kits, drip chambers, filters, burette chambers, stopcocks, multiport valves, vials, flow restriction valve, ‘Y’-sites and ‘T’-sites, sets (for example gravity sets, pump sets, specialty sets and the like), and ambulatory pumps, or valves of any design. Kits may include all of the various components needed for a given procedure. In this aspect, the kit may include at least one conversion device and other components known to one of ordinary skill.

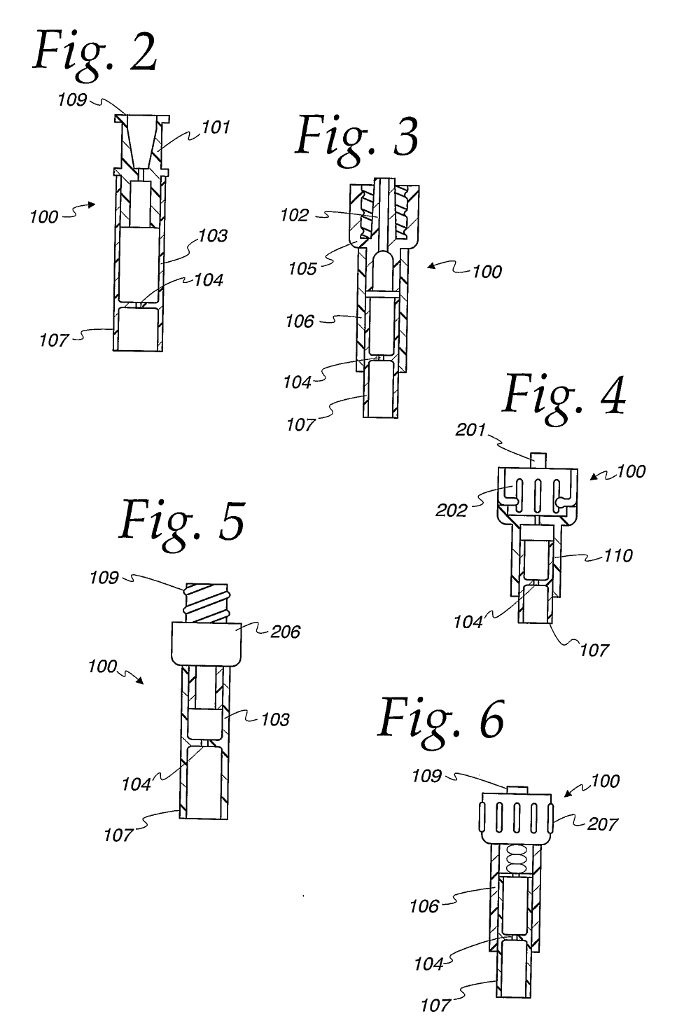

FIG. 2 illustrates one aspect of the conversion device 100 configured as connector/adaptor. The device includes female luer 101 on its distal end 109. The female luer 101 is attached to an integral tubular intermediate structure 103. Although the integral tubular intermediate structure 103 is attached to the outside of the female luer 101, one of ordinary skill would realize that the tubular intermediate structure 103 could be attached internally. A membrane port 107 is connected to the integral tubular intermediate structure 103. The membrane port 107 includes a membrane 104.

FIG. 3 illustrates another aspect of the conversion device 100. The device includes a male luer 102 which may include a collar 105. The collar 105 may be a rotatable collar. The male luer 102 is attached to a tubular intermediate structure 106. A membrane port 107 is connected to the integral tubular intermediate structure 103. The membrane port 107 includes a membrane 104.

FIG. 4 shows a male connection 201 in association with a bayonet attachment feature 202. The bayonet attachment feature 202 is associated with a receptacle for membrane port 110 which is configured to receive the membrane port 107.

FIG. 5 illustrates an ICU type female reflux valve 206 on a distal end 109 of the conversion device 100. The female reflux valve 206 is attached to a integral tubular intermediate structure 103, which is attached to a membrane port 107. Any female reflux valve may be utilized on the conversion device, including for example, female reflux valves available from B. Braun, Alaris, Haulkey-Roberts, Filtertec, Bola, Porex, and other known manufacturers.

FIG. 6 shows a conversion device 100 with a male reflux valve 207 on the distal end 109. The male reflux valve 207 is attached to a tubular intermediate structure 106 which is attached to a membrane port 107. Examples of male reflux valves that may be utilized include those described in U.S. Pat. Nos. 5,405,333, 5,645,538, 5,848,994, CA 2,171,857 and EP0719158, which are incorporated herein by reference.

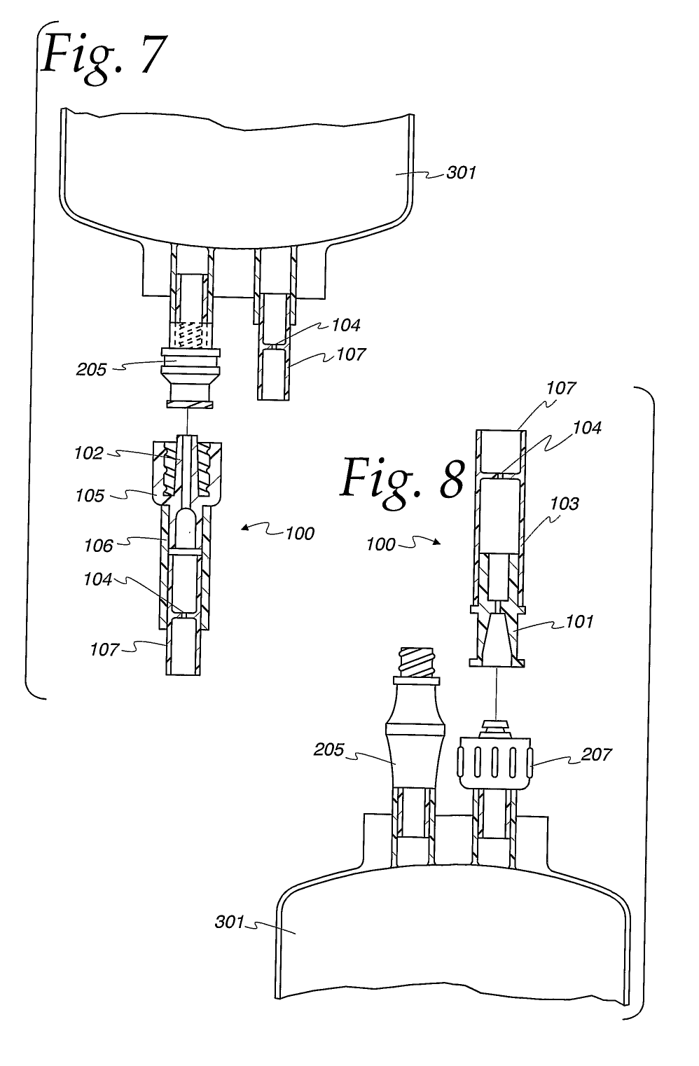

FIG. 7 illustrates a dual port container 301 that includes female reflux valve 205 and a membrane port 107. A conversion device may be attached to the female reflux valve 205 as shown. The conversion device 100 shown in FIG. 7 is similar to the conversion device 100 shown in FIG. 3.

FIG. 8 shows a dual port container 301 that includes at least one female reflux valve 205 and at least one male reflux valve 207. In this embodiment a conversion device 100 may be connected to the male reflux valve 207. The conversion device 100 shown in FIG. 8 is similar to the conversion device 100 shown in FIG. 2.

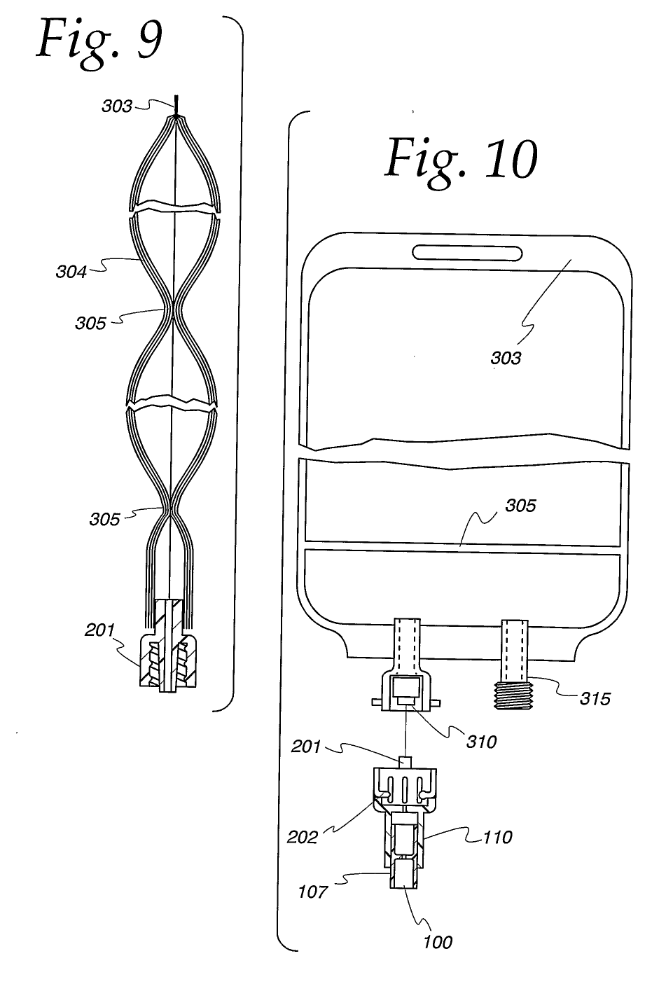

FIG. 9 illustrates multi-material walls 304 of a multi-material container 303. The container 303 includes tear seals 305 and a male connector 201. This type of container 303 may include at least two types of materials in the wall structure and at least one tear seal 305 depending on its end use. This container may include any number of ports.

FIG. 10 shows a multi-material container 303 that includes a tear seal 305, one port with a bayonet connector 202, and one port with a threaded female connector 315. The conversion port 100 may include a male connector 201 which connects with a port that includes a split septum 310. The threaded female connector 315 may be an open port.

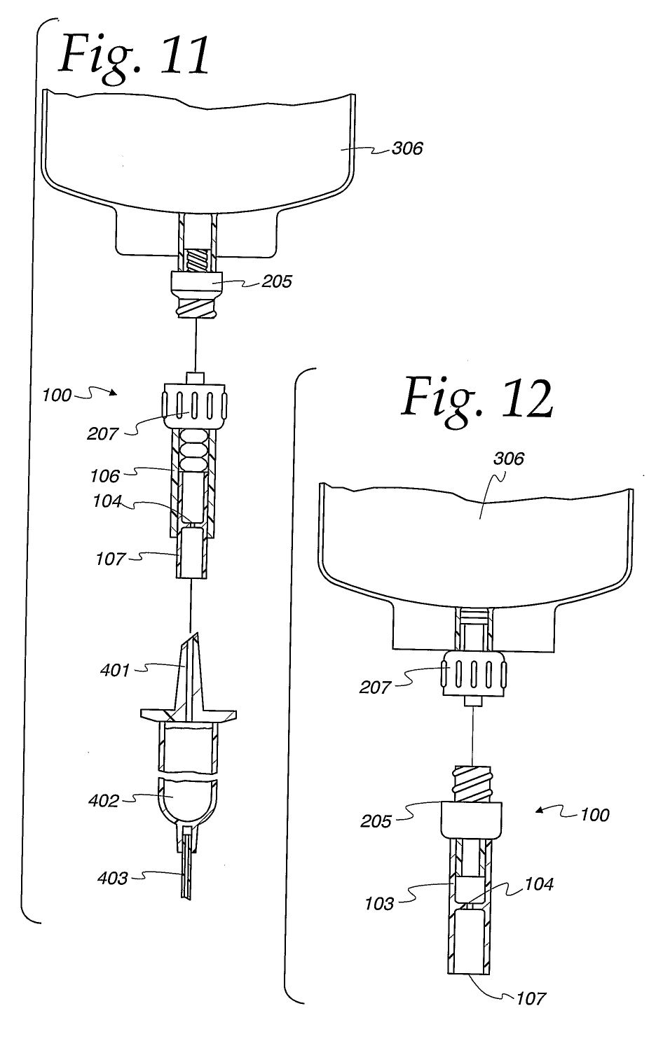

FIG. 11 illustrates a single port container 306 that may include a female reflux valve 205. As shown in the Figure, the conversion device 100, which is similar to the conversion device shown in FIG. 6, may be attached to the female reflux valve 205 on the container 306. In this embodiment, a drip chamber 402 having a spike 401 and set 403 may be brought into contact with the conversion device 101 to establish a flow of medicaments from the container 306, through the conversion device 100, through the drip chamber 402, and through the set 403. The container may include a belly button port, saddle port, boat port, or any known style of port.

FIG. 12 shows a single port container 306 that may include a male reflux valve 207. The conversion device 100 may include a female reflux valve 205.

FIG. 13 illustrates a conversion device 100 that includes a male luer 102 and collar 105 attached to a Y-intermediate structure 501. In the embodiment shown in the Figure, the Y-intermediate structure 501 connects to two membrane ports 107. One or both membrane ports 107 may be connected to a spike 401. The spike 401 may be further attached to tubing 405 which may be attached to a male fitting 407. Flow through the tubing 405 may be regulated with clamp 406.

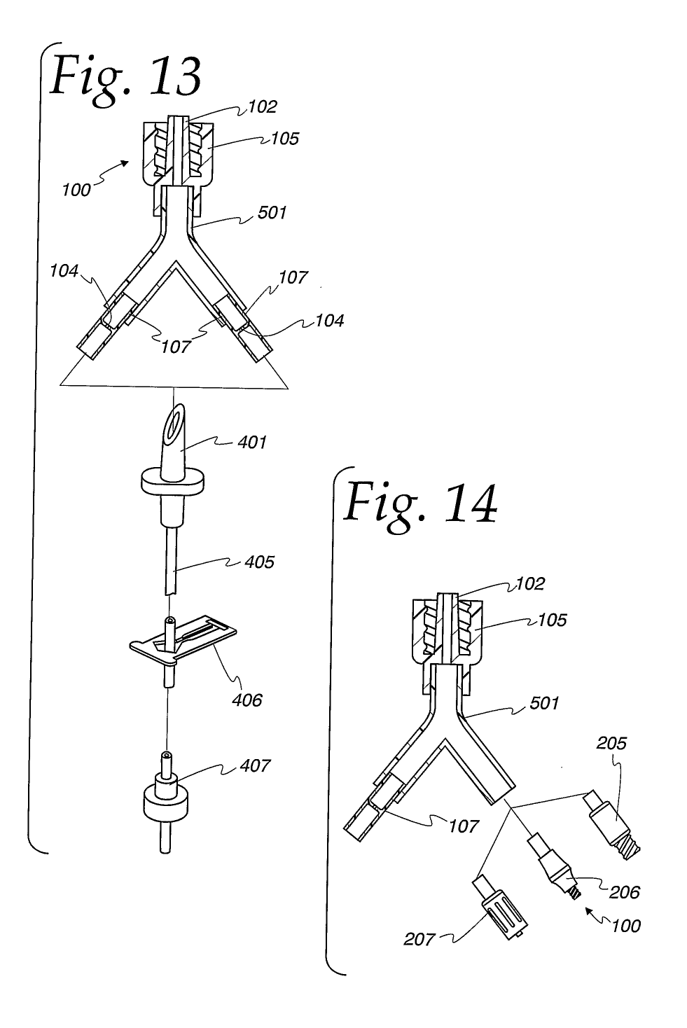

As further shown in FIG. 14, and as would be understood by one of ordinary skill, the Y-intermediate structure 501 may be attached to one membrane port 107 and the other arm of the Y-intermediate structure may be attached to any other type of connector, such as for example female reflux valves 205 and 206 or a male reflux valve.

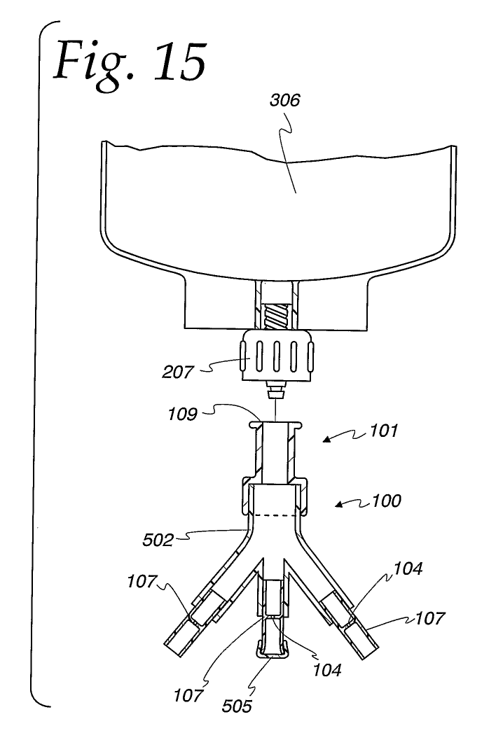

FIG. 15 illustrates a single port container 306 that may include a male reflux valve 207. The conversion device may include a female luer 101 on its distal end 109. The female luer 101 is connected to a junction body 502 that may lead to multiple branches. The branches may include one or more membrane ports 107. One of ordinary skill would realize that one or all of the branches may be configured as any type of connector as described herein. As shown in the Figure, another one of the branches from the junction body 502 may be an injection site 505.

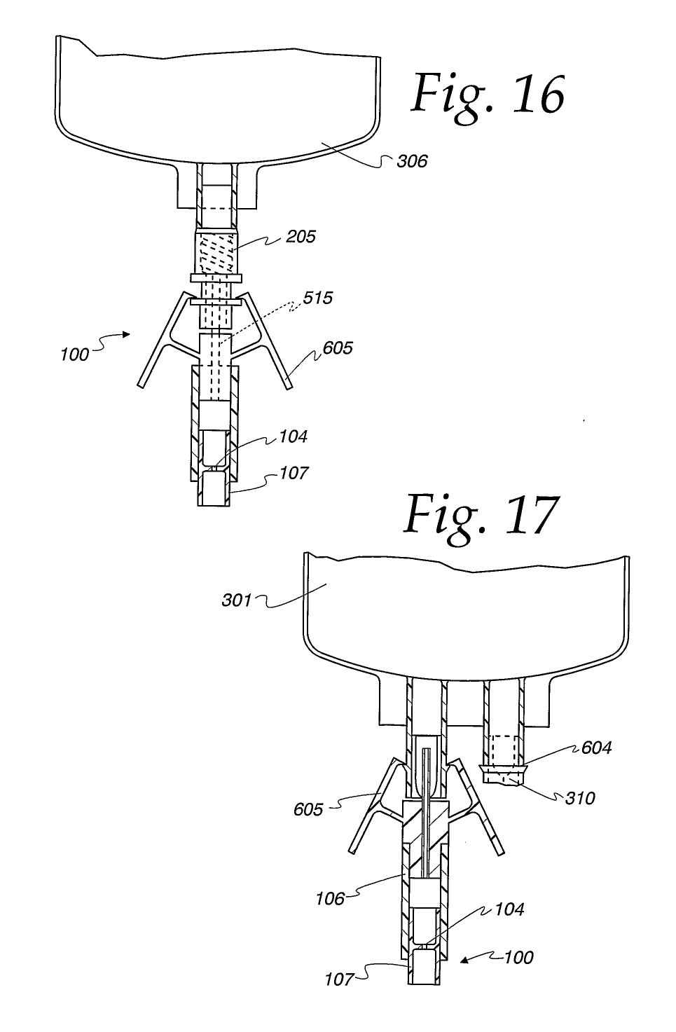

FIG. 16 shows a single port container 306 that includes a female reflux valve 205. The conversion device 100 shown in this embodiment includes a goal post style connector 605 and may further include a cannula 515.

FIG. 17 illustrates a dual port container 301 with a conversion device 100 similar to the conversion device 100 shown in FIG. 16 connected to one the ports. The second port includes a port with a split septum 310 and a retaining collar 604.

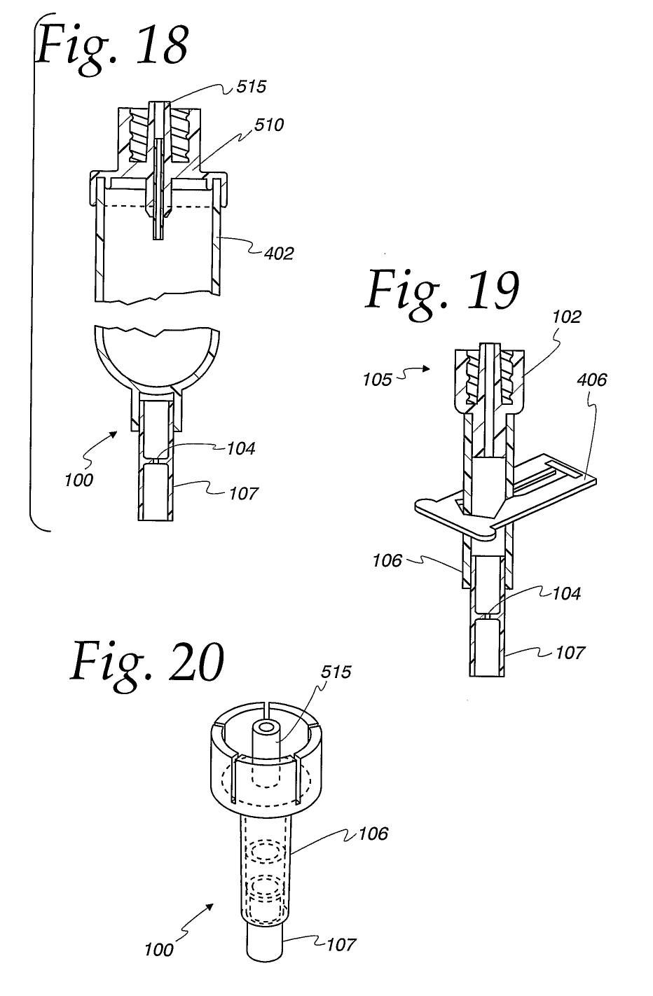

FIG. 18 shows a drip chamber 402 (one of many possible drip chamber configurations) that includes a drip chamber cap 510 and a cannula 515. In this embodiment, the drip chamber 402 may also be connected to a conversion device 100.

FIG. 19 illustrates a male luer 102 which may include a collar 105. The collar 105 may be a rotatable collar. The male luer 102 is attached to a tubular intermediate structure 106. Flow through the tubular intermediate structure 106 may be controlled with clamp 406. The clamp may be one of many possible designs of shut-off clamps. A membrane port 107 is connected to the integral tubular intermediate structure 106. The membrane port 107 includes a membrane 104.

FIG. 20 shows a conversion device 100 that includes a membrane port 107 connected to tubular intermediate structure 106 and a connector which includes a cannula 515 and a flexible split skirt 610.

This invention may be particularly useful for containers having a single port with a valve. The present invention is effective for allowing addition of or administration from container(s) having a single port with a valve as it allows for connection of a spike to the container having a single port with a valve.

Numerous modifications and variations in practice of the invention are expected to occur to those skilled in the art upon consideration of the foregoing detailed description of the invention. Consequently, such modifications and variations are intended to be included within the scope of the following claims.

What is claimed is:

1. A conversion device comprising an attachment feature effective for attaching to a second attachment feature, at least one membrane port on an end opposite the attachment feature, the membrane port effective for receiving a spike, and an intermediate structure disposed between the attachment feature and membrane port.

2. The conversion device of claim 1 wherein the attachment feature is selected from the group consisting of a male connector, female connector, cannula, male luer, male luer and collar, split skirt, female luer, bayonet connector, threads, partial threads, tabs, ears, male reflux valve, female reflux valve, goal post style connectors, flow restriction valve, or one-way valve.

3. The conversion device of claim 1 wherein the intermediate structure is selected from the group consisting of containers, fluid lines, tubing, connectors, sets, kits, drip chambers, filters, burette chambers, stopcocks, multiport valves, vials, flow restriction valve, ‘Y’-sites and ‘T’-sites, and ambulatory pumps.

4. The conversion device of claim 1 wherein the conversion device further comprises an injection site.

5. A method for connecting a spike to a valve comprising:
connecting a valve to an attachment feature on a conversion device; and
spiking a membrane port on the conversion device with a spike.

6. The method of claim 5 wherein the attachment feature is selected from the group consisting of a male connector, female connector, cannula, male luer, male luer and collar, split skirt, female luer, bayonet connector, threads, partial threads, tabs, ears, male reflux valve, female reflux valve, goal post style connectors, flow restriction valve, or one-way valve.

7. The method of claim 5 wherein the spike is attached to a medical system.

8. The method of claim 7 wherein the medical system is selected from the group consisting of a sets, kits, and ambulatory pumps.

9. A method for connecting a spiked medical system to a container comprising:
connecting a valve to an attachment feature on a conversion device; and
spiking a membrane port on the conversion device with a spike of the spiked medical system.

10. The method of claim 9 wherein the container includes at least one reflux valve.

11. The method of claim 9 wherein the attachment feature is selected from the group consisting of a male connector, female connector, cannula, male luer, male luer and collar, split skirt, female luer, bayonet connector, threads, partial threads, tabs, ears, male reflux valve, female reflux valve, goal post style connectors, flow restriction valve, or one-way valve.

12. The method of claim 9 wherein the spiked medical system is selected from the group consisting of a sets, kits and ambulatory pumps.
